(19) United States
(12) Patent Application Publication
(10) Pub. No.: US 2025/0288810 A1
(43) Pub. Date: Sep. 18, 2025

(54) SYSTEMS AND METHODS FOR COORDINATING ELECTROSTIMULATION AND MEDICATION FOR NEUROMODULATION THERAPY

(71) Applicant: Boston Scientific neuromodulation Corporation, Valencia, CA (US)

(72) Inventors: Mahsa Malekmohammadi, Sherman Oaks, CA (US); Lisa Denise Moore, Glendale, CA (US)

(21) Appl. No.: 19/068,273

(22) Filed: Mar. 3, 2025

Related U.S. Application Data
(60) Provisional application No. 63/566,627, filed on Mar. 18, 2024.

(51) Int. Cl. A61N 1/36; A61N 1/02; G16H 20/10; G16H 20/30

(52) U.S. Cl. CPC A61N 1/36135; A61N 1/025; G16H 20/10; G16H 20/30

(57) ABSTRACT

Systems and methods for coordinating electrostimulation and medication for neuromodulation therapy are disclosed. An exemplary system comprises a processing system. The processing system may use a model to determine neural tissue activated by medication administered to a patient. The processing system may determine a stimulation parameter from a stimulation parameter set that activates the neural tissue during delivery of electrostimulation. The processing system may map the neural tissue activated by the medication to the stimulation parameter. The processing system may select the stimulation parameter from the stimulation parameter set in response to the medication being associated with the patient.

### CLAIM OF PRIORITY

This application claims the benefit of U.S. Provisional Application No. 63/566,627, filed on Mar. 18, 2024, which is hereby incorporated by reference in its entirety.

### TECHNICAL FIELD

This document relates generally to medical systems, and more particularly, but not by way of limitation, to systems, devices, and methods for coordinating stimulation and medication for neuromodulation therapy.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc.

A therapy device may be configured to treat a condition. Thus, by way of example and not limitation, a DBS system may be configured to treat motor disorders such as, but not limited to, tremor, bradykinesia, and dyskinesia associated with Parkinson's Disease (PD). In another nonlimiting example, a stimulation device, such as neurostimulation device (e.g., DBS, SCS, PNS or TENS), may be configured to treat pain. In another nonlimiting example, a device, such as a myocardial stimulator and/or neurostimulator, may be configured to treat cardiovascular condition. Settings of the therapy device may be programmed based on observed clinical effects so that the therapy provides desirable intended effects (e.g., reduced tremor, bradykinesia, and dyskinesia for a PD therapy, desirable pain relief or paresthesia coverage for a pain therapy, desirable blood pressure and/rhythms for a cardiovascular therapy) while avoiding undesirable side effects.

### SUMMARY

An example (e.g., “Example 1”) of a system may include a processing system. The processing system may be configured for use to: use a model to determine neural tissue activated by medication administered to a patient; determine a stimulation parameter from a stimulation parameter set that activates the neural tissue during delivery of electrostimulation; map the neural tissue activated by the medication to a stimulation parameter set providing similar change in neural activity; and select the stimulation parameter from the stimulation parameter set in response to the medication being associated with the patient.

In Example 2, the subject matter of Example 1 optionally includes wherein the processing system is configured for use to program at least one medical device with the selected stimulation parameter to deliver the electrostimulation.

In Example 3, the subject matter of any one or more of Examples 1-2 optionally include wherein the processing system is configured to increase or decrease a medication dosage and increase or decrease a stimulation parameter of the stimulation parameter set based on a change in the activation of the neural tissue.

In Example 4, the subject matter of any one or more of Examples 1-3 optionally include wherein the processing system is configured for use to: determine a map of a stimulation clinical effect data (CED) based on the stimulation parameter set without administration of medication; and determine a map of the medication CED based on medicating the patient without applying stimulation.

In Example 5, the subject matter of any one or more of Examples 1-4 optionally include wherein the processing system is configured for use to generate the model based on at least one of pharmacodynamics, medication kinetics, an affect on neural pathways of the patient, patient medical history, and a symptom or side effect profile of the patient.

In Example 6, the subject matter of any one or more of Examples 1-5 optionally include wherein the processing system is configured for use to acquire medication data associated with the patient.

In Example 7, the subject matter of Example 6 optionally includes wherein the medication data comprises a medication type, a medication dosage amount, and pharmacodynamics associated with a medication for administering to the patient.

In Example 8, the subject matter of any one or more of Examples 6-7 optionally include wherein the processing system is configured for use to generate the model based on the medication data to simulate a medication state of the patient.

In Example 9, the subject matter of any one or more of Examples 6-8 optionally include wherein the medication data is acquired based on medications prescribed to the patient.

In Example 10, the subject matter of any one or more of Examples 6-9 optionally include wherein the medication data is acquired in response to dosing the patient and acquiring the medication data at a peak of medication dosage.

In Example 11, the subject matter of any one or more of Examples 1-10 optionally include wherein the processing system is configured for use to determine the stimulation parameter set based on a real-time medication state of the patient; wherein: the real-time medication state of the patient is an OFF state of medication of the patient; and the stimulation parameter set indicates a particular stimulation parameter associated with the OFF state.

In Example 12, the subject matter of any one or more of Examples 1-11 optionally include wherein the processing system is configured for use to increase a stimulation parameter of the stimulation parameter set in response to medication data indicating that a medication dosage of the patient is decreasing.

In Example 13, the subject matter of any one or more of Examples 1-12 optionally include wherein the processing system is configured for use to decrease a stimulation parameter of the stimulation parameter set in response to medication data indicating that medication dosage of the patient is increasing.

In Example 14, the subject matter of any one or more of Examples 1-13 optionally include wherein the processing system is configured for use to determine a map of medication CED based on the mapping of the activated neural tissue and by comparing a medication ON state to a medication OFF state in an absence of applying stimulation.

In Example 15, the subject matter of Example 14 optionally includes wherein the map of the medication CED and a map of stimulation CED are used to provide a recommendation of medication and stimulation parameters to a physician.

Example 16 includes subject matter (such as a method, means for performing acts, machine readable medium including instructions that when performed by a machine cause the machine to perform acts, or an apparatus to perform). The subject matter may include using a model to determine neural tissue of a patient activated by medication administered to a patient. The subject matter may include determining a stimulation parameter from a stimulation parameter set that activates the neural tissue during delivery of electrostimulation. The subject matter may include mapping the neural tissue activated by the medication to a stimulation parameter set providing similar change in neural activity of the patient. The subject matter may include selecting the stimulation parameter from the stimulation parameter set in response to the medication being associated with the patient.

In Example 17, the subject matter of Example 16 optionally includes using the mapping to determine whether to increase or decrease a medication dosage and increase or decrease a stimulation parameter of the stimulation parameter set.

In Example 18, the subject matter of any one or more of Examples 16-17 optionally include determining: a map of stimulation clinical effect data (CED) based on the stimulation parameter set without administration of medication; and a map of medication CED based on medicating the patient without applying stimulation.

In Example 19, the subject matter of Example 18 optionally includes wherein the map of the medication CED is simulated based on the neural tissue activated by the medication.

In Example 20, the subject matter of Example 19 optionally includes wherein the map of the medication CED and the neural tissue activated is correlated to equivalent anatomical locations of electrical stimulation.

In Example 21, the subject matter of any one or more of Examples 18-20 optionally include wherein the map of medication CED is an estimated map of medication CED that is estimated for a medication state without the patient being in the medication state.

In Example 22, the subject matter of any one or more of Examples 18-21 optionally include wherein the map of stimulation CED is an estimated map of stimulation CED that is estimated for a medication state without the patient being in the medication state.

In Example 23, the subject matter of any one or more of Examples 18-22 optionally include adjusting the map of the medication CED in response to a period of time occurring associated with a decrease in medication effectiveness post-administration.

In Example 24, the subject matter of any one or more of Examples 18-23 optionally include adjusting the map of the stimulation CED in response to a period of time occurring associated with a decrease or increase in medication effectiveness.

In Example 25, the subject matter of any one or more of Examples 18-24 optionally include wherein the determining of the map of the medication CED is performed without having the patient washing out of medication.

In Example 26, the subject matter of any one or more of Examples 16-25 optionally include generating the model of neural tissue activated by medication based on at least one of pharmacodynamics, medication kinetics, an affect on neural pathways of the patient, patient medical history, and a symptom or side effect profile of the patient.

In Example 27, the subject matter of any one or more of Examples 16-26 optionally include acquiring medication data associated with the patient.

In Example 28, the subject matter of Example 27 optionally includes wherein the medication data comprises a medication type, a medication dosage amount, and pharmacodynamics associated with a medication for administering to the patient.

In Example 29, the subject matter of any one or more of Examples 27-28 optionally include wherein the model is generated based on the medication data to simulate a medication state of the patient.

In Example 30, the subject matter of any one or more of Examples 27-29 optionally include wherein the medication data is acquired based on medications prescribed to the patient.

In Example 31, the subject matter of any one or more of Examples 27-30 optionally include wherein the medication data is acquired in response to dosing the patient and acquiring the medication data at a peak of medication dosage.

Example 32 includes subject matter (such as a method, means for performing acts, machine readable medium including instructions that when performed by a machine cause the machine to perform acts, or an apparatus to perform). The subject matter may include using a number of models to each determine a set of neural tissue of a patient activated by a corresponding medication. The subject matter may include determining a stimulation parameter from a stimulation parameter set that activates each of the corresponding neural tissue. The subject matter may include generating a number of maps that each map the neural tissue activated by the corresponding medications to their respective stimulation parameters. The subject matter may include selecting respective stimulation parameters from the stimulation parameter set in response to the corresponding medications being associated with the patient. The subject matter may include adjusting the respective stimulation parameters based on a determined schedule for the medications.

In Example 33, the subject matter of Example 32 optionally includes wherein the instructions are executable by the processor to determine whether to increase or decrease a medication dosage and increase or decrease a stimulation parameter of the stimulation parameter set based on the number of maps.

In Example 34, the subject matter of any one or more of Examples 32-33 optionally include wherein the instructions are executable by the processor to generate: a map of a stimulation CED based on the stimulation parameter set without administration of medication; and a map of the medication CED based on medicating the patient without applying stimulation.

In Example 35, the subject matter of Example 34 optionally includes wherein the instructions are executable by the processor to determine a level of adjustment of the stimulation parameter set that correlates with a transition from a medication low state to a medication high state.

Example 36 includes subject matter (such as a method, means for performing acts, machine readable medium including instructions that when performed by a machine cause the machine to perform acts, or an apparatus to perform). The subject matter of Example 36 may include at least one medical device and a processing system. The subject matter may include determining a first stimulation parameter of a stimulation parameter set for delivering electrostimulation during a high medication state of the patient. The subject matter may include determining a second stimulation parameter of the stimulation parameter set for delivering the electrostimulation during a low medication state of the patient. The subject matter may include adjusting the stimulation parameter set from the first stimulation parameter when the patient is in the high medication state to the second stimulation parameter when the patient is in the low medication state.

In Example 37, the subject matter of Example 36 optionally includes wherein the processing system adjusting the parameter set from the first stimulation parameter to the second stimulation parameter comprises increasing an amplitude for delivering the electrostimulation.

In Example 38, the subject matter of any one or more of Examples 36-37 optionally include wherein the processing system is configured to deliver the electrostimulation using the first stimulation parameter in response to occurrence of a set wash-in time for a scheduled medication dose.

In Example 39, the subject matter of any one or more of Examples 36-38 optionally include wherein the processing system is configured to deliver the electrostimulation using an intermediate stimulation parameter between the first stimulation parameter and the second stimulation parameter subsequent to an occurrence of a set wash-in time.

In Example 40, the subject matter of Example 39 optionally includes wherein the processing system is configured to gradually and linearly increase a stimulation parameter used to deliver electrostimulation as the patient transitions from the high medication state to the low medication state.

In Example 41, the subject matter of Example 40 optionally includes wherein the processing system is configured to transition the stimulation parameter from the high medication state to the low medication state during a period of time based on a medication schedule.

In Example 42, the subject matter of Example 41 optionally includes wherein the processing system is configured to transition the stimulation parameter during each successive period of time associated with each successive medication dosage is adjusted based on an accumulation affect of taking multiple dosages.

In Example 43, the subject matter of any one or more of Examples 36-42 optionally include wherein the processing system is configured to deliver the electrostimulation using the second stimulation parameter in response to occurrence of a set wash-out time for a scheduled medication dose.

In Example 44, the subject matter of any one or more of Examples 36-43 optionally include wherein the first stimulation parameter is a lower amplitude than the second stimulation parameter.

In Example 45, the subject matter of any one or more of Examples 36-44 optionally include wherein the processing system is configured to adjust the parameter set from the second stimulation parameter to the first stimulation parameter in response to a second dose of medication being administered and the patient transitioning from a medication wash-out to a medication wash-in state.

In Example 46, the subject matter of any one or more of Examples 36-45 optionally include wherein the processing system is configured to use a default stimulation parameter during a period of sleep of the patient.

In Example 47, the subject matter of Example 46 optionally includes wherein the processing system is configured to deliver the electrostimulation based on a transition from a high medication state to a low medication state in response to the patient entering or nearing a wake state.

In Example 48, the subject matter of any one or more of Examples 36-47 optionally include wherein the processing system is configured to receive from a user, in order to perform delivery the therapy, at least one of: a medication low state value: a medication high state value; a default stimulation parameter; a patient's medication schedule; and a selected medication wash-in time.

In Example 49, the subject matter of Example 48 optionally includes wherein the processing system is configured to recommend a stimulation schedule based on data received from the patient.

In Example 50, the subject matter of any one or more of Examples 48-49 optionally include wherein the processing system is configured to receive an instruction from the patient to switch to a different stimulation program or change an amplitude used for the delivery of the electrostimulation.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example, an example of an electrical therapy-delivery system.

FIG. 6 illustrates, by way of example and not limitation, a monitoring system and/or the electrical therapy-delivery system of FIG. 5, implemented using an implantable medical device (IMD).

FIG. 7A illustrates, by way of example and not limitation, a method of coordinating electrostimulation and medication for neuromodulation therapy.

FIG. 7B illustrates, by way of example and not limitation, a method of coordinating electrostimulation and medication for neuromodulation therapy.

FIG. 8 illustrates, by way of example and not limitation, a method of coordinating electrostimulation and medication for neuromodulation therapy.

FIG. 9 illustrates, by way of example and not limitation, a flowchart of a method of coordinating electrostimulation and medication for neuromodulation therapy.

FIG. 10 illustrates, by way of example and not limitation, a flowchart of a method of coordinating electrostimulation and medication for neuromodulation therapy.

FIGS. 11A-11D each illustrate, by way of example and not limitation, an amplitude of the electrostimulation that corresponds to a medication for neuromodulation therapy.

FIG. 12 illustrates, by way of example and not limitation, an example of a schedule including varying amplitudes that correspond to periods of time for administering neuromodulation therapy.

FIG. 13 illustrates, by way of example and not limitation, an example of a schedule including varying amplitudes that correspond to periods of time for administering neuromodulation therapy.

FIG. 14 illustrates, by way of example and not limitation, a neuromodulation therapy system, which may be used to deliver DBS.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Movement disorders are often treated with a combination of medication and stimulation. For example, patients can be implanted with a deep brain stimulation (DBS) implant and also administered medication to alleviate symptoms. During a 24 hour cycle of taking medications, depending on their pharmacodynamics and/or pharmacokinetics, medication concentrations can wax and wane over time and their therapeutic efficacy can change accordingly, resulting in a high state of medication or a low state of medication. Physicians can balance treatments of electrostimulation and/or medication so that when the patient is in a low state of medication, the system adequately addresses the patient's symptoms by supplementing with electrostimulation. Likewise, when the patient is in a high state of medication the electrostimulation provided to the patient can be adjusted (e.g., lowered) so that the patient is not experiencing excessive electrostimulation and/or unwanted side effects.

In some examples, being off medication can cause parkinsonism and being on medication with overlapping electrostimulation can cause dyskinesia. In some prior approaches, the electrostimulation can be programmed while the patient is in a medication OFF state (e.g., where a period of time is used to allow the medication to wash out of the patient, causing further delays and consumes time) and the electrostimulation program can be adjusted after administering the medication to the patient. Other complications can include dyskinesia not necessarily developing with a single dose of medication and using two or more doses in order for the dyskinesia to manifest. The prior approach of washing medication out and performing electrostimulation adjustments while the medication washes back in can result in a compromise for each of the therapies, e.g., for each of the electrostimulation or the medication, and may not be optimal for either of the therapies and their combination. Further, medications may be gradually adjusted to a lower dose over time but how to concurrently adjust the electrostimulation to compensate has not been shown previously.

As described herein, a translation between the effects of medication and the effects of electrostimulation can be used to avoid time-consuming schedules and adjustments and to more closely approximate optimal therapies for using both electrostimulation and medication. Such a translation of effects between medication and electrostimulation can include using a basic understanding of the mechanism of action (MOA) and/or pharmacodynamics and kinetics for medications and how they affect different neural pathways, in addition to the patient's medical history and symptoms or side effect profile. The MOA and pharmacodynamics of the medication can be translated to an equivalent electrostimulation of different shapes, locations, and/or sizes that best interacts with the corresponding neural pathways to estimate a medication clinical effect data (CED) map. The medication CED map can be combined with a CED map for the electrostimulation to be used to generate the overall therapy. Further, as described herein, an automated or manual schedule of electrostimulation and medication is generated using the patient's known medication states (e.g., high states, low states, etc.) to titrate stimulation based on the estimated dosing. Further, a patient's medication HIGH states and medication LOW states may be used to determine the patient's medication schedule to create an automated or manual schedule to titrate stimulation based on the estimated dosage of medication.

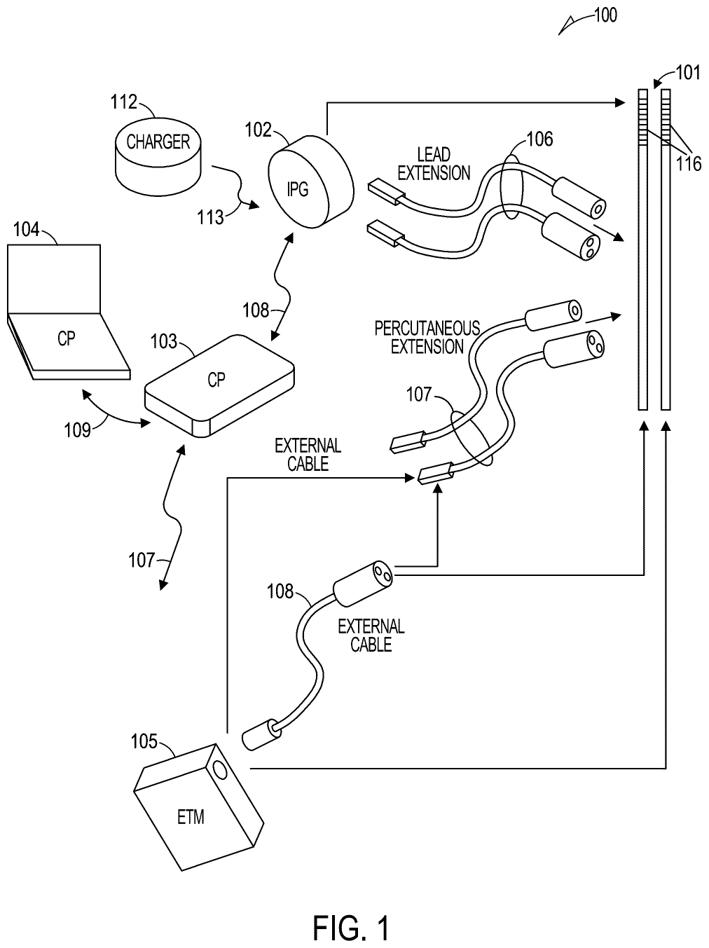

FIG. 1 illustrates, by way of example and not limitation, an electrical stimulation system 100, which may be used to deliver DBS. The electrical stimulation system 100 may generally include one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads may be provided, including only one, as long as the number of electrodes is greater than two (including the IPG case function as a case electrode) to allow for lateral steering of the current. Alternatively, a surgical paddle lead may be used in place of one or more of the percutaneous leads. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 may likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters may be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies may be performed by executing software instructions contained within the CP 104. Alternatively, such programming methodologies may be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneously charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

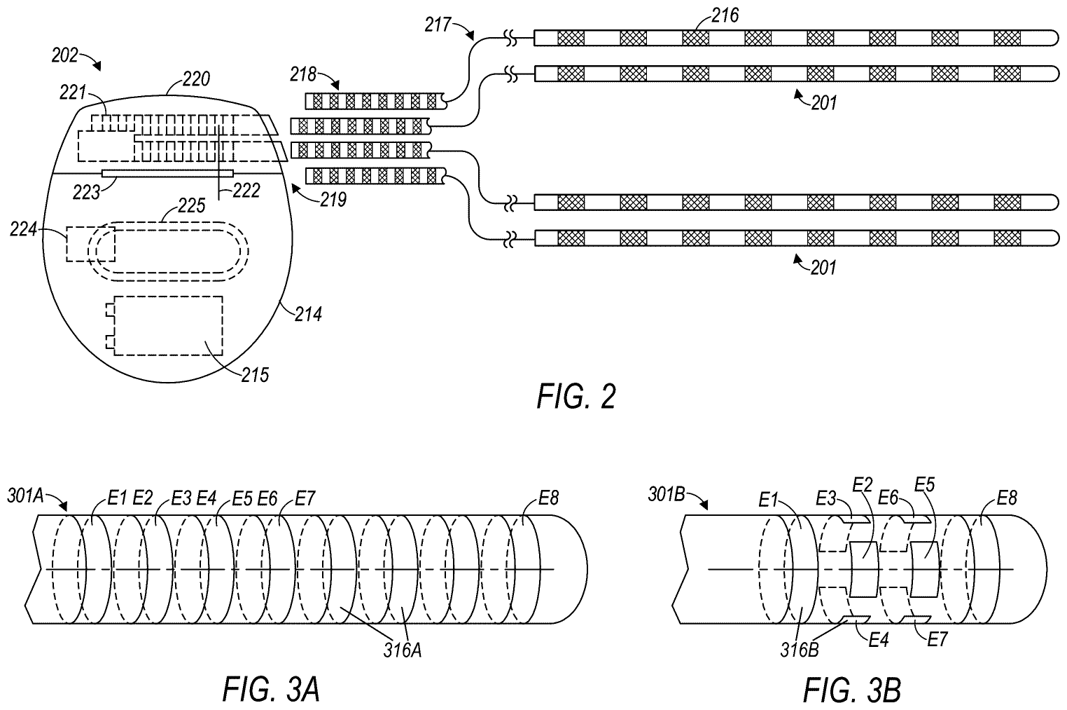

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 may also lack a battery and may be wirelessly powered by an external source. The IPG 202 may be coupled to one or more leads, such as leads 201 as illustrated herein. The leads 201 may each include a plurality of electrodes 216 for delivering electrostimulation energy, recording electrical signals, or both. In some examples, the leads 201 may be rotatable so that the electrodes 216 may be aligned with the target neurons after the neurons have been located such as based on the recorded signals. The electrodes 216 may include one or more ring electrodes, and/or one or more rows of segmented electrodes (or any other combination of electrodes), examples of which are discussed below with reference to FIGS. 3A and 3B.

The leads 201 may be implanted near or within the desired portion of the body to be stimulated. In an example of operations for DBS, access to the desired position in the brain may be accomplished by drilling a hole in the patient's skull or cranium with a cranial drill (commonly referred to as a burr), and coagulating and incising the dura mater, or brain covering. A lead may then be inserted into the cranium and brain tissue with the assistance of a stylet (not shown). The lead may be guided to the target location within the brain using, for example, a stereotactic frame and a microdrive motor system. In some examples, the microdrive motor system may be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header may comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore may vary.

The IPG 202 may include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a radiofrequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) may be tunneled through and under the neck and the scalp, with the electrodes 216 implanted through holes drilled in the skull and positioned for example in the subthalamic nucleus (STN) in each brain hemisphere. The IPG 202 may also be implanted underneath the scalp closer to the location of the electrodes' implantation. The leads 201, or the extensions, may be integrated with and permanently connected to the IPG 202 in other solutions.

Stimulation in IPG 202 is typically provided by pulses each of which may include one phase or multiple phases. For example, a monopolar stimulation current may be delivered between a lead-based electrode (e.g., one of the electrodes 216) and a case electrode. A bipolar stimulation current may be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue; a comparison of a set threshold value; user-determined values or thresholds, or a change/delta from a prior measurement. Each of the electrodes may either be used (an active electrode) or unused (OFF). When the electrode is used, the electrode may be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 may execute to provide therapeutic stimulation to a patient.

In some examples, a measurement device coupled to the muscles or other tissue stimulated by the target neurons, or a unit responsive to the patient or clinician, may be coupled to the IPG 202 or microdrive motor system. The measurement device, user, or clinician may indicate a response by the target muscles or other tissue to the stimulation or recording electrode(s) to further identify the target neurons and facilitate positioning of the stimulating electrode(s). For example, if the target neurons are directed to a muscle experiencing tremors, a measurement device may be used to observe the muscle and indicate changes in, for example, tremor frequency or amplitude in response to stimulation of neurons. Alternatively, the patient or clinician may observe the muscle and provide feedback.

FIGS. 3A-3B illustrate, by way of example and not limitation, leads that may be coupled to the IPG to deliver electrostimulation such as DBS. FIG. 3A shows a non-directional lead 301A with electrodes 316A disposed at least partially about a circumference of the non-directional lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a directional lead 301B with electrodes 316B including ring electrodes such as E1 at a proximal end and E8 at the distal end. Additionally, the lead 301 also includes a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 may direct stimulus current to a selected angular range around the lead.

Segmented electrodes may typically provide more-superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering may be performed not only along a length of the lead but also around a circumference of the lead. This provides precise three-dimensional targeting and delivery of the current stimulus to neural target tissue, while potentially avoiding stimulation of other tissue. In some examples, segmented electrodes may be together with ring electrodes. A lead which includes at least one or more segmented electrodes may be referred to as a directional lead. In an example, all electrodes on a directional lead may be segmented electrodes. In another example, there may be different numbers of segmented electrodes at different longitudinal positions.

Segmented electrodes may be grouped into rows of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all rows of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The rows of segmented electrodes may be positioned in irregular or regular intervals along a length of the lead 201.

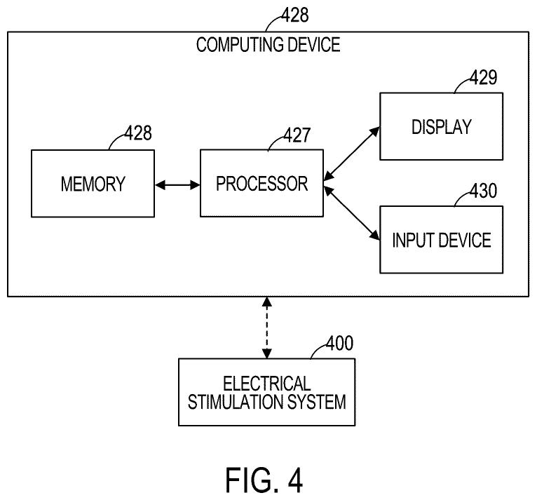

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1 Alternatively, the computing device 426 may be integrated with the electrical stimulation system 100, such as part of the IPG 102, RC 103, CP 104, or ETM 105 illustrated in FIG. 1. The computing device may be used to perform process(s) for sensing parameter(s).

The computing device 426, also referred to as a programming device, may be a computer, tablet, mobile device, or any other suitable device for processing information. The computing device 426 may be local to the user or may include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 may include a watch, wristband, smartphone, or the like. Such computing devices may wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease.

In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 may be a wearable device used by the patient only during programming sessions. Alternatively, the computing device 426 may be worn all the time and continually or periodically adjust the stimulation parameters. In an example, a closed-loop algorithm for determining or updating stimulation parameters may be implemented in a mobile device, such as a smartphone, which is connected to the IPG or an evaluating device (e.g., a wristband or watch). These devices may also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit may be a dedicated processor such as a digital signal processor, application specific integrated circuit (ASIC), microprocessor, or other type of processor for processing information. Alternatively, the microprocessor circuit may be a processor that may receive and execute a set of instructions of performing the functions, methods, or techniques described herein.

The memory 428 may store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which may be used to store the desired information, and which may be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and may include a printer. The display 429 may be a part of a user interface configured to display information about stimulation settings (e.g., electrode configurations and stimulation parameter values and value ranges) and user control elements for programming a stimulation setting into an IPG. The computing device 426 may include other output(s) such as speaker(s) and haptic output(s) (e.g., vibration motor).

The input device 430 may be, for example, a keyboard, mouse, touch screen, track ball, joystick, voice recognition system, or any combination thereof, or the like. Another input device 430 may be a camera from which the clinician may observe the patient. Yet another input device 430 may a microphone where the patient or clinician may provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user may provide information between the electrical stimulation system 400 and the computing device 426 using a computer-readable medium or by some other mechanism.

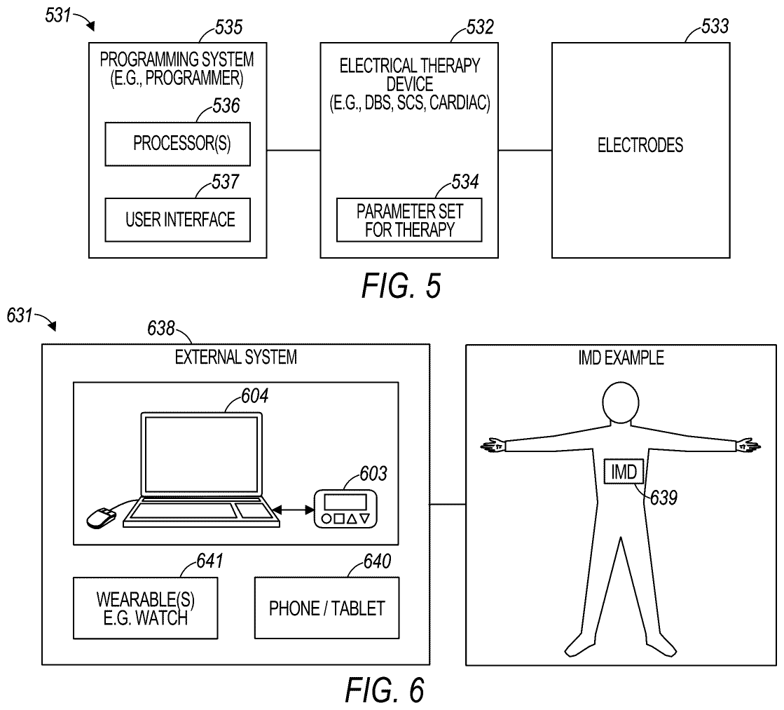

FIG. 5 illustrates, by way of example, an example of an electrical therapy-delivery system. The illustrated system 531 includes an electrical therapy device 532 configured to deliver an electrical therapy to electrodes 533 to treat a condition in accordance with a programmed parameter set 534 for the therapy. The system 531 may include a programming system 535, which may function as at least a portion of a processing system, which may include one or more processors 536 and a user interface 537. The programming system 535 may be used to program and/or evaluate the parameter set(s) used to deliver the therapy. The illustrated system 531 may be a DBS system.

In some embodiments, the illustrated system 531 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameter sets through a computerized programming system to allow the optimum modulation parameters to be determined based on patient feedback or other means and to subsequently program the desired modulation parameter sets.

FIG. 6 illustrates, by way of example and not limitation, a monitoring system and/or the electrical therapy-delivery system of FIG. 5, implemented using an implantable medical device (IMD). The illustrated system 631 includes an external system 638 that may include at least one programming device. The illustrated external system 638 may include a clinician programmer 604, similar to CP 104 in FIG. 1, configured for use by a clinician to communicate with and program the neuromodulator, and a remote control device 603, similar to RC 103 in FIG. 1, configured for use by the patient to communicate with and program the neuromodulator. For example, the remote control device 603 may allow the patient to turn a therapy on and off, change or select programs, and/or may allow the patient to adjust patient-programmable parameter(s) of the plurality of modulation parameters. FIG. 6 illustrates an IMD 639, although the monitor and/or therapy device may be an external device such as a wearable device. The external system 638 may include a network of computers, including computer(s) remotely located from the IMD 639 that are capable of communicating via one or more communication networks with the programmer 604 and/or the remote control device 603. The remotely located computer(s) and the IMD 639 may be configured to communicate with each other via another external device such as the programmer 604 or the remote control device 603. The remote control device 603 and/or the programmer 604 may allow a user (e.g., patient and/or clinician or rep) to answer questions as part of a data collection process. The external system 638 may include personal devices such as a phone or tablet 640, wearables such as a watch 641, sensors or therapy-applying devices. The watch may include sensor(s), such as sensor(s) for detecting activity, motion and/or posture. Other wearable sensor(s) may be configured for use to detect activity, motion and/or posture of the patient. The external system 638 may include, but is not limited to, a phone and/or a tablet. Notifications may be sent to the patient, physician, device rep or other users via the external system and through remote portals (e.g., web-based portals) provided by remote systems.

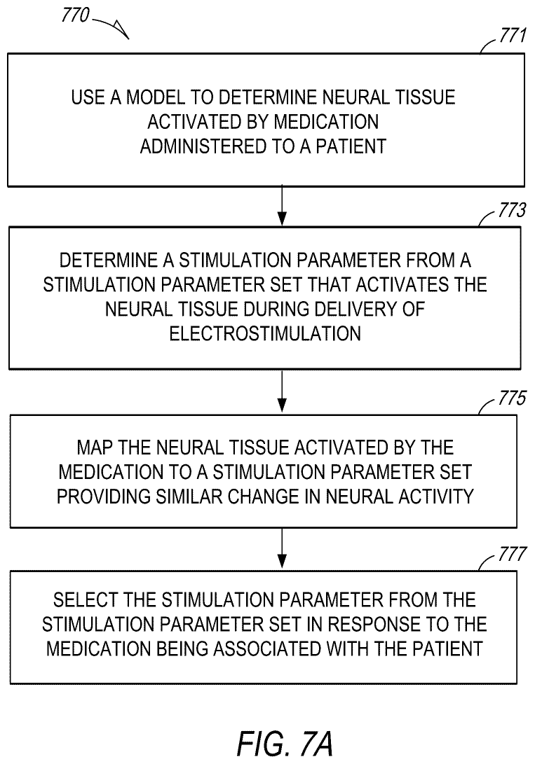

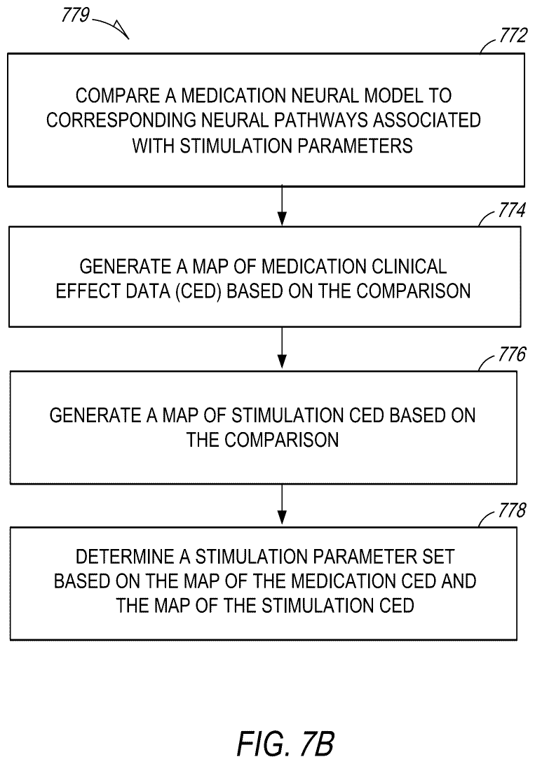

FIG. 7A illustrates, by way of example and not limitation, a method 770 of coordinating electrostimulation and medication for neuromodulation therapy. At 771, the method 770 can include using a model to determine neural tissue activated by medication administered to a patient. The method 770 can include generating the model based on at least one of pharmacodynamics, medication kinetics, an affect on neural pathways of the patient or function measured by various modalities of neuroimaging such as functional magnetic resonance imaging (fMRI), electroencephalography (EEG), Magnetoencephalography (MEG), Positron Emission Tomography (PET) or Optical Imaging, patient medical history, or a symptom or side effect profile of the patient. The model can be generated based on the medication data to simulate a medication state of the patient. The model of neural tissue activated by medication can be generated based on at least one of pharmacodynamics, medication kinetics, an affect on neural pathways of the patient, patient medical history, and a symptom or side effect profile of the patient.

At 773, the method 770 can include determining a stimulation parameter from a stimulation parameter set that activates the neural tissue during delivery of electrostimulation. The method 770 can include determining the stimulation parameter set based on a real-time medication state of the patient. The real-time medication state of the patient can be an OFF state of medication of the patient. The stimulation parameter set can indicate a particular stimulation parameter associated with the OFF state.

At 775, the method 770 can include mapping the neural tissue activated by the medication to a stimulation parameter set providing similar change in neural activity. The method 770 can further include determining a map of a stimulation clinical effect data (CED) based on the stimulation parameter set without administration of medication. The method 770 can further include determining a map of the medication CED based on medicating the patient without applying stimulation. The method 770 can further include determining a map of medication CED based on the mapping of the activated neural tissue and by comparing a medication ON state to a medication OFF state in an absence of applying electrostimulation. The determining of the map of the medication CED can be performed without having the patient washing out of medication. The map of medication CED can be an estimated map of medication CED that is predicated on a medication state without the patient being in the medication state. The map of the medication CED and the neural tissue activated can be compared to equivalent anatomical locations of electrical stimulation. The map of the medication CED may be adjusted in response to a period of time occurring associated with a decrease in medication effectiveness post-administration or doe response curves associated with nonlinear or non-monotonous dynamics.

The method can further include determining a map of stimulation clinical effect data (CED) based on the stimulation parameter set without administration of medication and determining a map of medication CED based on medicating the patient without applying stimulation. The map of stimulation CED is an estimated map of stimulation CED that is estimated for a medication state without the patient being in the medication state. The map of the medication CED and a map of stimulation CED can be used to provide a recommendation of medication and stimulation parameters to a physician. The mapping of the neural tissue activated by the medication to the stimulation parameter can be used to determine whether to increase or decrease a medication dosage and change a stimulation parameter of the stimulation parameter set. The map of the stimulation CED can be adjusted in response to a period of time occurring associated with a decrease or increase in medication effectiveness or concentration.

At 777, the method 770 can include selecting the stimulation parameter from the stimulation parameter set in response to the medication being associated with the patient. The method 770 can include programming at least one medical device with the selected stimulation parameter to deliver the electrostimulation. The method 770 can further include changing a stimulation parameter of the stimulation parameter set in response to medication data indicating that a medication dosage of the patient is decreasing. The method 770 can further include changing a stimulation parameter of the stimulation parameter set in response to medication data indicating that medication dosage of the patient is increasing.

The method 770 can further include increasing or decreasing a medication dosage and increase or decrease a stimulation parameter of the stimulation parameter set based on a change in the activation of the neural tissue. The method 770 can further include acquiring medication data associated with the patient. The medication data can include a medication type, a medication dosage amount, pharmacodynamics associated with a medication for administering to the patient, relative timing of the medication, and absolute timing of the medication. The medication data can be acquired based on medications prescribed to the patient. The medication data can be acquired in response to dosing the patient and acquiring the medication data at a peak of medication dosage or in response to modeling based on a medication type and pharmacodynamic information associated with the medication type.

The method 770 can include generating a number of maps (such as a medication CED map and/or a stimulation CED map) that each map the neural tissue activated by the corresponding medications to their respective stimulation parameters. The method 770 can include adjusting the respective stimulation parameters based on a determined schedule for the medications. The method 770 can include determining whether to increase or decrease a medication dosage and increase or decrease a stimulation parameter of the stimulation parameter set based on the number of maps. The method 770 can include determining a level of adjustment of the stimulation parameter set that correlates with a transition from a medication low state to a medication high state.

FIG. 7B illustrates, by way of example and not limitation, a method 779 of coordinating electrostimulation and medication for neuromodulation therapy. At 772, the method 779 can include comparing a medication neural model to corresponding neural pathways associated with stimulation parameters. At 774, the method 779 can include generating a map of medication clinical effect data (CED) based on the comparison. The map of medication CED can include data estimated for medication alone using the translation of the neural pathways described above or gathered from the patient during a period of administration of medication alone (e.g., without administering electrostimulation).

At 776, the method 779 can include generating a map of stimulation CED based on the comparison. The map of the stimulation CED can include data estimated for stimulation alone using the translation of the neural pathways described above or gathered from the patient during a period of administration of stimulation alone (e.g., without administering medication or during a period where the medication has washed out or worn off). The map of the medication CED may be simulated for medications by translating the effects of medications on different neural circuits and translating those back into equivalent anatomical electrical stimulation of different parts of the stimulation parameters. For example, the effects of the medication Levodopa on reducing tremors can be modeled as potentially stimulating a tremor sweet spot with respect to anatomy with the size of a stimulation field model (SFM) corresponding to a degree of activation of the sweet spot based on correlations with a degree of improvement in the tremors. Further, the effects of levodopa on dyskinesia may be modeled as potentially stimulating a dyskinesia sour spot with respect to anatomy with the size of the SFM corresponding to a degree of activation of the sour spot based on correlations with a degree of worsening in the dyskinesia. The effects of medication on non-motor symptoms such as compulsive disorders may be mapped to stimulation of the limbic or associative sub-thalamic nucleus (STN). At 778, the method 779 can include determining a stimulation parameter set based on the map of the medication CED and the map of the stimulation CED.

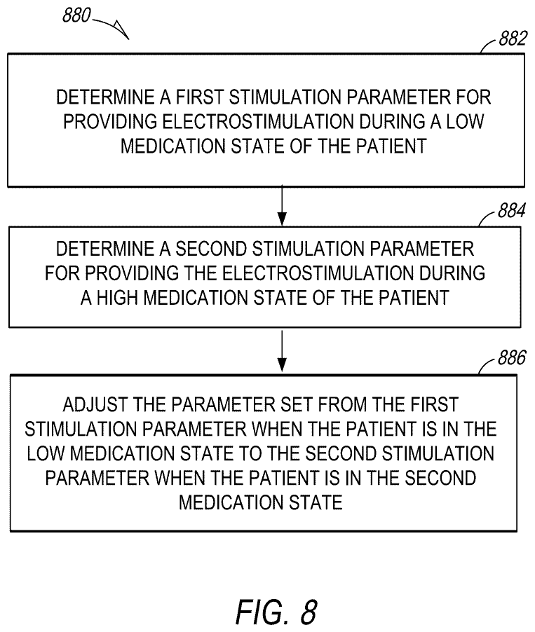

FIG. 8 illustrates, by way of example and not limitation, a method 880 of coordinating electrostimulation and medication for neuromodulation therapy. The method 880 may be performed in association with machine readable medium including instructions that, when performed by the machine cause the machine to perform acts, or an apparatus to perform the method. At least one medical device and/or a processing system may be used to perform the method. At 882, the method 880 can include determining a first stimulation parameter for providing electrostimulation during a low medication state of the patient.

At 884, the method 880 may include determining a second stimulation parameter of the stimulation parameter set for delivering the electrostimulation during a low medication state of the patient. At 886, the method 880 may include adjusting the stimulation parameter set from the first stimulation parameter when the patient is in the high medication state to the second stimulation parameter when the patient is in the low medication state. Adjustment of the parameter set from the first stimulation parameter to the second stimulation parameter may include increasing an amplitude for delivering the electrostimulation. The method 880 may include adjusting the parameter set from the second stimulation parameter to the first stimulation parameter in response to a second dose of medication being administered and the patient transitioning from a medication wash-out to a medication wash-in state.

The method 880 may include delivering the electrostimulation using the first stimulation parameter in response to occurrence of a set wash-in time for a scheduled medication dose. The method 880 may include delivering the electrostimulation using an intermediate stimulation parameter between the first stimulation parameter and the second stimulation parameter subsequent to an occurrence of a set wash-in time. A stimulation parameter used to deliver electrostimulation as the patient transitions from the high medication state to the low medication state may be gradually and linearly increased. The method 880 may include transitioning the stimulation parameter from the high medication state to the low medication state during a period of time based on a medication schedule. The stimulation parameter may be transitioned during each successive period of time associated with each successive medication dosage is adjusted based on an accumulation affect of taking multiple dosages.

The method 880 may include delivering the electrostimulation using the second stimulation parameter in response to occurrence of a set wash-out time for a scheduled medication dose. The first stimulation parameter may be a lower amplitude than the second stimulation parameter. The method 880 may include using a default stimulation parameter during a period of sleep of the patient. The method 880 may include delivering the electrostimulation based on a transition from a high medication state to a low medication state in response to the patient entering or nearing a wake state. The method 880 may include receiving from a user, in order to perform delivery the therapy, at least one of: a medication low state value: a medication high state value; a default stimulation parameter; a patient's medication schedule; and a selected medication wash-in time. The method 880 may include recommending a stimulation schedule based on data received from the patient. The method 880 may include receiving an instruction from the patient to switch to a different stimulation program or change an amplitude used for the delivery of the electrostimulation.

A medication state of the patient may be determined based on the at least one feature. As an example, a high medication state, a medium medication state, a low medication state, etc., may be determined based on the feature. The high medication state may indicate a high level of medication in the patient, a high absorption rate of the medication, and/or a high efficacy of the medication on the patient, where each of these examples indicates the patient's symptoms are highly alleviated or the medication is being more highly effective. The high level, absorption rate, effectiveness, etc., may be in relation to a medium or lower level, rate, or effectiveness based on many data points related to the patient. A medium medication state may indicate a medium level of medication in the patient, a medium absorption rate of the medication, and/or a medium efficacy of the medication on the patient, where each of these examples indicates the patient's symptoms are alleviated or the medication is effective to a moderate degree. The medium absorption rate, effectiveness, etc., may be in relation to a higher or lower rate or effectiveness based on many data points related to the patient. Likewise, the low medication state may indicate a low level of medication in the patient, a low absorption rate of the medication, and/or a low effectiveness of the medication on the patient, where each of these examples indicates the patient's symptoms are alleviated or the medication is being more effective to a lower degree than other levels, rates, or efficacies. The medication state can be an absolute medication state or a relative medication state. For example, the absolute medication state can be a medication state independent of other factors whereas a relative medication state can be a medication including comparisons of or effects from other factors. In some examples, the medication state may be considered on a continuum with extreme values being high and low and in-between states being calculated as between the high and low states.

Medication state may be estimated based on a patient's medication schedule or based on the exact times that patients are administered the medication. However, patients may forget to take the medication, may forget to take the medication using the correct medication schedule, may metabolize the medication at different rates (as the metabolization rates are patient specific), the medication may fail to have a proper effect (e.g., rendered ineffective), or absorption of the medication may be affected by dietary intake. For example, with respect to dietary intake, when the food is eaten, how much, what kind of food or drink was consumed may affect the medication. In addition, an affect of the medication can be altered based on a medication tablet failing to be processed in the stomach of the patient or absorbed in the stomach and/or intestines. Based on estimates, patient specific medication metabolism rates may be estimated. Further, periods where wearing off exceeds a specific threshold may be identified. Medication schedule adjustments may be recommended based on these estimations. Anomalies in patient medication for the determined state compared to the time of dose taken may be used to identify factors affecting the medication or preventing the medication from taking effect or full effect. As an example, eating certain foods prior to taking medication may reduce the efficacy of certain medications.

While the above description refers to a medication state, a state of a patient may also be used to determine or adjust medication levels or electrostimulation levels. The state of the patient may be determined based on whether a value for the at least one feature is within a defined range of values. The state may be determined to be a high state in response to the value being within a first threshold range of values. The state may be determined to be a low state in response to the value being within a second threshold range of values. The state may be determined to be an optimal state in response to the value being associated with a particular patient outcome. The state may be determined to be an average state in response to the value being within a third threshold range associated with a most common range of the at least one feature for a set period of time. The high state, the low state, the optimal state, and the average state may each be patient specific.

Based on the at least one feature, a state of the patient may be determined from a sleep state, an awake state, or a state of physical activity. The sleep state may be associated with a particular level of sleep, such as rapid eye movement (REM) sleep, or a particular stage of sleep (e.g., a first, second, third, or fourth stage of sleep). The awake state may be associated with a particular type of awake state such as an alert state, a focused state, a fatigued state, etc. The state of physical activity may be associated with a level of physical activity such as a low level, a medium level, and/or a high level that each indicate an intensity of activity being performed or recently performed by the body of the patient and/or other factors.

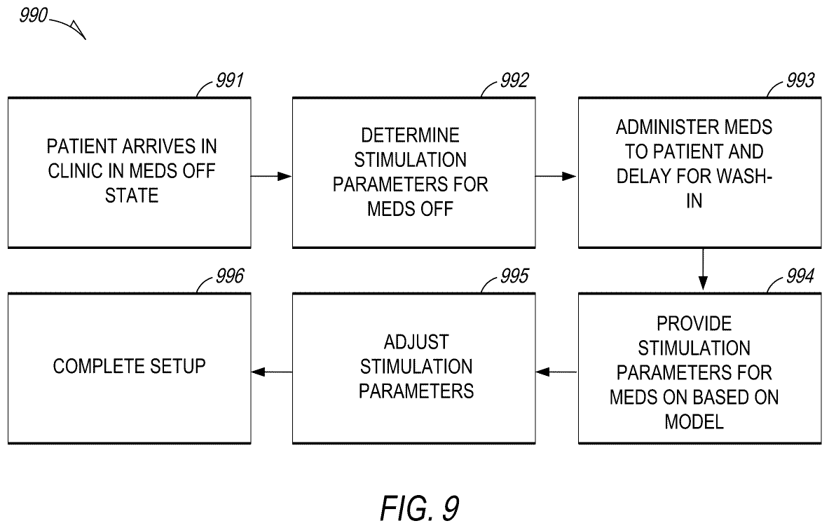

FIG. 9 illustrates, by way of example and not limitation, a flowchart 990 of a method of coordinating electrostimulation and medication for neuromodulation therapy. At 991, the flowchart 990 may include a patient arriving in a clinic in a medication OFF state. For example, the patient may arrive at the clinic subsequent to medication washing out or wearing off. In response to the patient arriving at the clinic with medication that has not yet worn off, the patient may wait a period of time in the clinic while the medication washes out or wears off.

At 992, the flowchart 990 may include determining stimulation parameters for a medication OFF state of the patient. For example, in response to the medication being washed out or wearing off, a series of stimulation parameters can be tested in order to determine which stimulation parameters most effectively provide effective or optimal therapy in the absence of medication.

At 993, the flowchart 990 may include administering medication to the patient and delaying proceeding until the medication has washed-in. As an example, particular symptoms or testing values may be associated with the medication being washed in and once those symptoms, or alleviation of those symptoms, is achieved, the medication administered to the patient can be considered to have washed-in or become fully effective.

At 994, the flowchart 990 may include providing stimulation parameters for medication based on a neural model. The stimulation parameters can be provided to a clinician, a representative, a doctor, etc., for review and approval to be administered or selected for subsequent therapy.

At 995, the flowchart 990 may include adjusting the stimulation parameters. The stimulation parameters may be adjusted based on differing symptoms and/or response from real-time data collected from the patient as the stimulation parameters are applied to the patient. The stimulation parameters may be adjusted based on revisions to the neural model and/or revisions to the map of the medication CED and/or the map of the stimulation CED. At 996, the flowchart 990 may include completing the setup. Once the setup is complete, the stimulation parameters and associated programming data may be stored in the medical device and/or stored by the provider in a database.

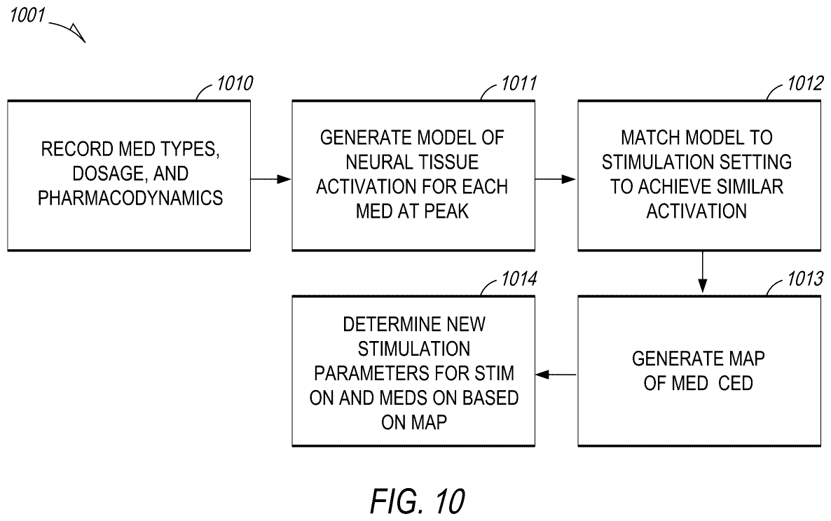

FIG. 10 illustrates, by way of example and not limitation, a flowchart 1001 of a method of coordinating electrostimulation and medication for neuromodulation therapy. At 1010, the flowchart 1001 may include recording medication types, dosage, and pharmacodynamics. The medication type can include one of: Carbidopa-Levodopa, Carbidopa-Levodopa entacapone, Levodopa Inhalation powder, Entacapone, Tolcapone, Opicapone, Carbidopa/Levodopa Entacapone, Praxipexole, Ropinirole, Apomorphine, Apomorphine sublingual film, Rotigotine (transdermal patch), Selegiline, Rasagiline, Safinamide, Amantadine, Istradefylline, Trihexyphenidyl, Benzotropine, among other medications, as this is not an exhaustive list and may include other medications. The medication type may indicate which portion of neural tissue is activated. The dosage may indicate how strongly or how broadly the portion of neural tissue is activated. The pharmacodynamics may indicate how the portion of neural tissue is activated or which corresponding stimulation parameters may most effectively match with the portion of neural tissue that is activated.

At 1011, the flowchart 1001 may include generating a model of neural tissue activation for each medication at a peak level. The model of neural tissue activation can be generated based on the medication type, the medication dosage, and/or the pharmacodynamics of the medication to be administered to the patient.

At 1012, the flowchart 1001 may include matching the model to a stimulation setting to achieve similar activation. For example, the stimulation setting that is matched may be selected due to the stimulation setting causing activation of the same neural tissue that is activated by the medication. In this way, the medication may have a same effect as the stimulation and therefore each of the medication and the stimulation can be matched and/or translated to one another. When the medication is decreased and the activation of the neural tissue is therefore decreased, the matched stimulation parameter may be increased to cause a commensurate increase in activation at the neural tissue. Vice versa, as the medication is increased, the stimulation parameter can be decreased, and so forth.

At 1013, the flowchart 1001 may include generating a map of a medication CED. The map of the medication CED may be generated by combining a group of estimated SFMs for the medication(s) and the level of rating scale improvements, e.g., improvements of the Unified Parkinson's disease rating scale (UPDRS), for an ON state of the medication and/or an ON only state of medication (where the patient is in an OFF state with respect to stimulation).

At 1014, the flowchart 1001 may include determining new stimulation parameters for stimulation ON and medication ON based on the map of the medication CED. The map of the medication CED may be a new weight map used to determine the new stimulation parameters. The medication alone CED map may be derived from the anatomy and outcomes testing could define the Isolated Medication Effected. This medication alone CED map may be determined pre-implant and prior to stimulation occurring. The symptoms and selected weights for the map of the stimulation CED alone could be set by comparing the Medication ON state to the Medication OFF state (with stimulation being in the OFF state in both cases). A map of the stimulation CED alone (e.g., with Medication OFF) could be derived from a combination of outcomes testing and the corresponding anatomy could define the Isolated Stimulation Effect (which could be performed at initial programming). With the map of the medication CED alone, a shift could be predicted that would occur as a patient transitions from a medication LOW state to a medication HIGH state. This shift could set the symptoms and weights to query the map of the stimulation CED alone and dynamically adjust the stimulation parameters to have the same effect or outcome as a known or estimated medication state.

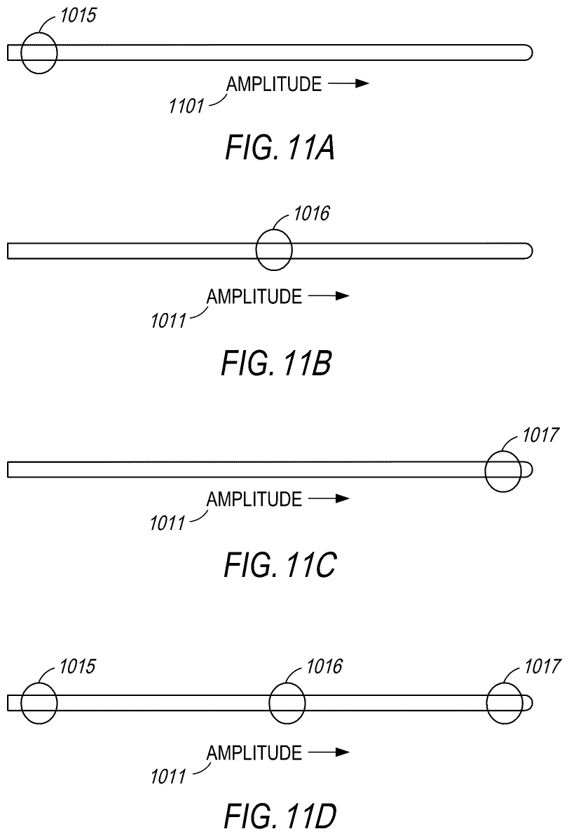

FIGS. 11A-11D each illustrate, by way of example and not limitation, an amplitude of the electrostimulation that corresponds to a medication for neuromodulation therapy. As shown in each of FIGS. 11A-11D, the amplitude 1101 increases from left to right, as illustrated and shown by an arrow. A physician may use the CP, such as CP 103 in FIG. 1, or other programming tool to calibrate the medical device by identifying the best stimulation parameters as well as a stimulation range (i.e., range of amplitude) for the stimulation parameters to provide to the patient during therapy. The physician may mark where on the amplitude range that stimulation should be applied during different medication states (e.g., during a medication LOW state, a medication HIGH state, etc.).

As shown in FIG. 11A, a low amplitude value 1015 may be associated with a HIGH medication state. As shown in FIG. 11B, a medium amplitude value 1016 may be associated with a default setting or a medium (“MED”) medication state. The default setting may be used for an effective chronic setting. As shown in FIG. 11C, a high amplitude value 1017 may be associated with a LOW medication state. FIG. 11D illustrates all three of the low amplitude value 1015, medium amplitude value 1016, and high amplitude value 1017 used by a physician or therapy provider to determine which corresponding medication states to apply which amplitude values for providing electrostimulation therapy. The physician may then confirm the patient's medication schedule and set an anticipated medication wash in time. In some examples, the wash in time can be between 20-40 minutes. The wash in time can refer to a period of time that the medication takes from administration to a peak saturation in the body of the patient, e.g., the time period for the medication to have a maximum effect on the patient's body.

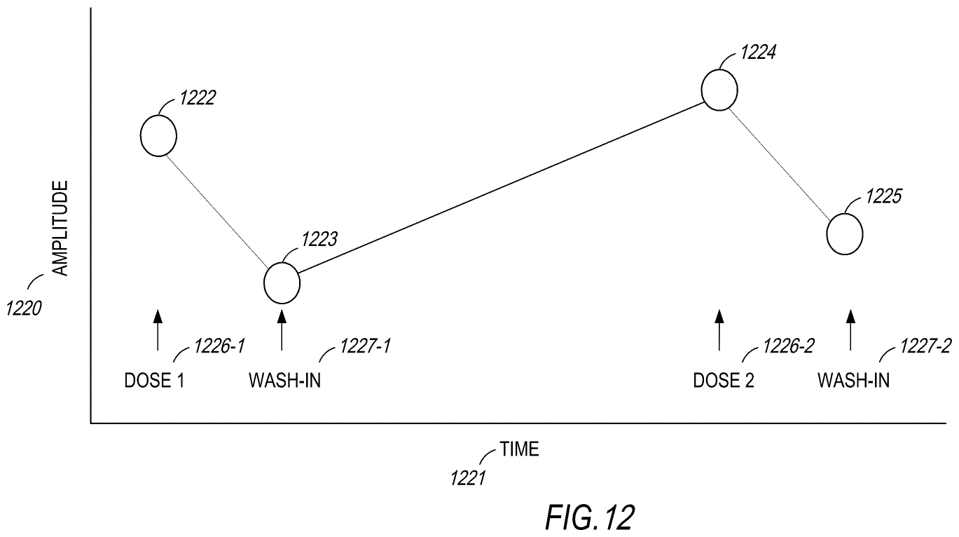

FIG. 12 illustrates, by way of example and not limitation, an example of a schedule including varying amplitudes that correspond to periods of time for administering neuromodulation therapy. The x-axis can refer to the time 1221 of the schedule and the y-axis can refer to an amplitude 1220 used during the time periods of the schedule. At the time of a first dosage (“DOSE 1”) 1226-1, the medication state is LOW and therefore the amplitude 1222 at that point is higher. At the time of the wash-in 1227-1 of the medication, the amplitude 1223 is lower as the medication is at a HIGH state. From the wash-in 1127-1 to a second dose (“DOSE 2”) 1226-2, the amplitude is gradually increased to an amplitude 1224 that is higher. That is, the amplitude is gradually increased as the medication gradually decreases as it wears off or washes out. As the medication is being washed in from the second dosage 1226-2, the amplitude is decreased from amplitude 1224 to amplitude 1225, at which point the medication is washed-in 1227-2.

This cyclical pattern of increasing the amplitude while the medication is washing out and decreasing the amplitude as the medication washes in can be repeated for each dosage cycle. The higher amplitudes of 1222 and 1224 can be set from the determined amplitude of 1017 in FIG. 11C and FIG. 11D. The lower amplitudes of 1123 and 1225 can be set from the determined amplitude of 1015 in FIG. 11A and FIG. 11D. The stimulation schedule can be set based on the medication schedule. In some examples, the increasing and decreasing of the stimulation parameters can take into account the accumulative impact of taking medication multiple times throughout a day and can set a window for change based on the scheduled dosages. For example, in this scenario, the medication in the lowest state may be when the patient wakes up in the morning since no dosage accumulation has occurred. The medication in the highest state may be the last dosage of the day that the patient takes as this dosage would have the greatest accumulation of dosages throughout the day.

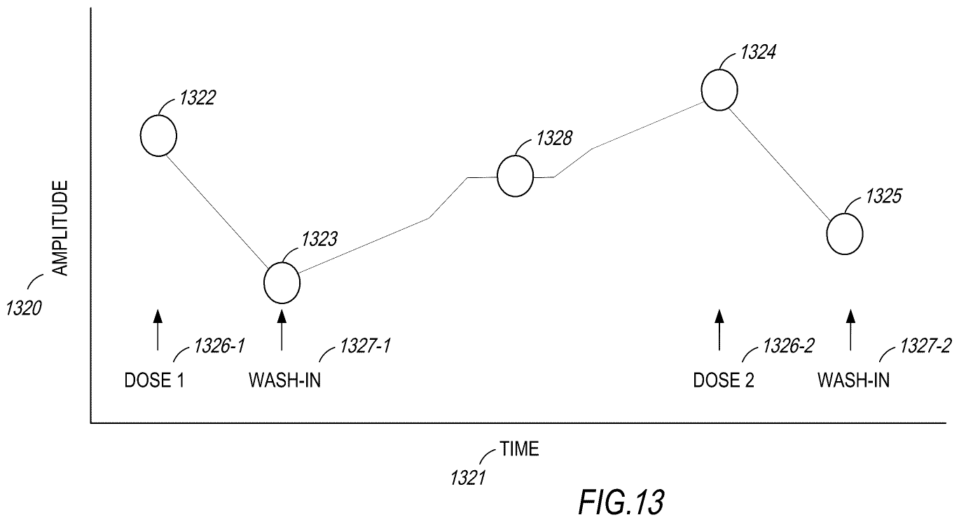

FIG. 13 illustrates, by way of example and not limitation, an example of a schedule including varying amplitudes that correspond to periods of time for administering neuromodulation therapy. Similar to FIG. 12, the x-axis can refer to the time 1321 of the schedule and the y-axis can refer to an amplitude 1320 used during the time periods of the schedule. FIG. 13 illustrates a similar setup to FIG. 12 in that the amplitude 1322 is decreased from the first dosage (“DOSE 1”) 1326-1 to the amplitude 1323 at the wash-in point 1327-1. Likewise, the amplitude 1324 is decreased from the time of the second dose (“DOSE 2”) 1326-2 to the amplitude 1325. However, during the patient's average sleep time when the state of the symptoms are different due to patients experiencing less motor symptoms, the amplitude 1328 can be set at a default setting (such as default amplitude 1016 in FIG. 11B) and can be ramped up from the amplitude 1323 prior to the sleep time occurring. Further, prior to the patient waking up from sleep, the system may transition back to the next window, such as ramp back up to amplitude 1324, to ensure an effective therapy is delivered when the patient is awake.

In some examples, for a stimulation parameter where a user has established a range and implements a medication adjustment scheduled change, several workflows may be used. A basic workflow may include a user using a user interface that indicates a medication HIGH, medication LOW, or medication default position. The user may be asked to enter a patient's medication schedule and select a wash in time. The system may recommend a particular schedule based on the inputs from the user and other analysis. A second, advance workflow may include a user indicating in a user interface the medication HIGH, medication LOW, or medication default position. The user may be asked to enter the patient's medication schedule and select a wash in time. The user may set windows of stimulation for each dose timing. Based on the input entered, the schedule for the stimulation programming may be automatically generated. The schedule may be run based on the internal IPG timer with a regular interval of adjustment based on the actual time recorded by any connected device that can synchronize with the current time.

Automated adjustments for the patient can include a patient or caregiver changing the medication schedule or sleep schedule within an application which can be used to update the schedule. The medication, wash in times, or sleep schedule may be updated based on manually or automatically collected information that may recommend a change. In the event that the patient is traveling and the time has changed significantly from the previously collected time point, the user may be prompted to reconfirm their medication schedule. The system may then adjust therapy to match any revisions. Further, titration amplitudes within set windows can be adapted based on feedback from the patient or caregiver or internal or externally sensed outcomes. In the event that therapy delivery is consistently delivering substandard outcomes at medication LOW points and additional range is available on the system, the lower amplitudes may be auto-adjusted to higher amplitudes. The same may be true if side effects are observed during medication HIGH points and stimulation may be decreased. In response to a patient making manual adjustments during a set time period, a schedule change may be triggered. Instances where the schedule may have underperformed can be recorded and used to recommend changes, automate changes, or be displayed to any user (e.g., a patient, a caregiver, a physician, etc.). Any of the examples described above may be done automatically or as part of a recommendation sent to the patient, caregiver, or physician on an at home or in clinic system.

In some examples, in response to the physician or caregiver switching between two programs as part of the scheduling, the user may use a more advanced mode. For example, the user ma set times for program switching as well as how the transition from one program to another may occur. In response to the patient switching to another program or changing the amplitude, the change may be allowed without restriction. When a peak treatment change would have occurred, the patient may be asked if the patient would like to resume a prior schedule. While all prior examples demonstrated a scheduled linear ramp up, the curve of such ramp-ups may be altered based on prior data, recommendations, or advanced user settings.

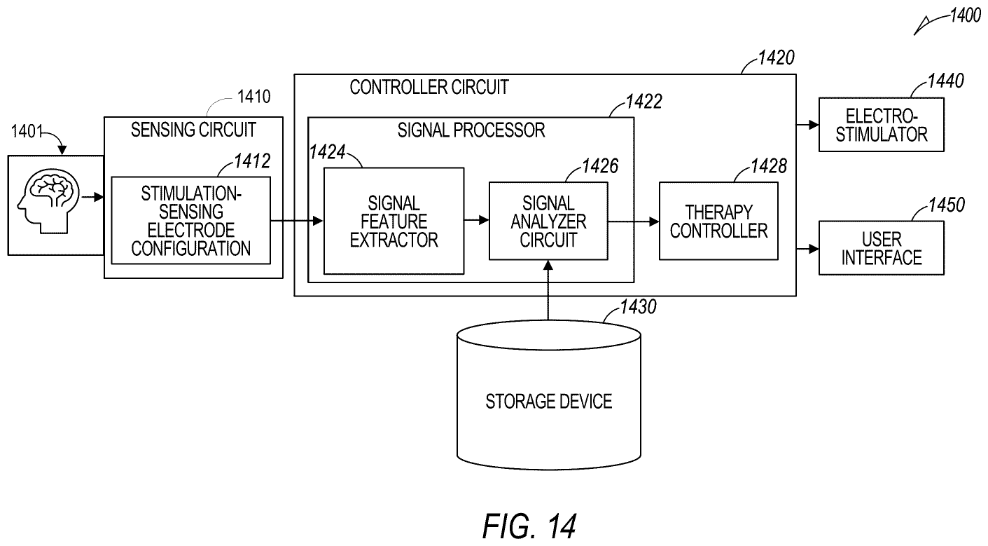

FIG. 14 illustrates, by way of example and not limitation, a neuromodulation therapy system, which may be used to deliver DBS based on evoked responses. The system 1400 includes a sensing circuit 1410, a controller circuit 1420, a storage device 1430, an electrostimulator 1440, and a user interface 1450. Portions of the system 1400 may be implemented in the IPG 102 or the CP 104.

The sensing circuit 1410 may be operatively connected to one or more leads and electrodes associated therewith, such as ring electrodes or segmented electrodes on the non-directional lead 301A or the directional lead 301B. The ring electrodes and/or the segmented electrodes may also be electrically coupled to the electrostimulator 1440. The ring electrodes and/or the segmented electrodes may be configured as sensing electrodes for sensing ERs, or as stimulating electrodes for delivering electrostimulation pulses. The sensing circuit 1414 may sense ERs from one or more sensing electrodes on a lead placed at target issue (e.g., STN) of a patient 1401 in response to electrostimulation pulses delivered from a stimulating electrode at a stimulation site (e.g., a brain target). The ERs may be sensed in accordance with a stimulating-sensing electrode configuration 1412.

The controller circuit 1420 may include circuit sets comprising one or more other circuits or sub-circuits, such as a signal processor 1422 and a therapy controller 1428. The signal processor 1422 may further include a signal feature extractor 1424 and a signal analyzer circuit 1426. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation.

In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

In various examples, portions of the functions of the controller circuit 1420 may be implemented as a part of a microprocessor circuit. The microprocessor circuit may be a dedicated processor such as a digital signal processor, application specific integrated circuit (ASIC), microprocessor, or other type of processor for processing information including physical activity information. Alternatively, the microprocessor circuit may be a general purpose processor that may receive and execute a set of instructions of performing the methods or techniques described herein.

The signal feature extractor 1424 may extract a signal feature from the filtered ER signal (e.g., the ER signal with the stimulation artifact removed). Examples of the signal features may include a signal amplitude, magnitude, peak value, value range, a signal curve length, or a signal power or RMS value of an ER signal within a time window, such as the epoch-averaged ERs. The signal amplitude range or value range, also referred to as a peak-to-peak (P2P) value, may be measured as a difference between a maximum value or a minimum value of a dominant peak in the sensed evoked response or an epoch-averaged evoked response within the time window (also referred to as “max P2P” amplitude). Alternatively, the P2P value may be measured as a difference between a negative peak (trough) and an immediate subsequent positive peak (also referred to as “N1-P2 P2P” amplitude). The signal curve length may be measured as accumulated signal value differences of the sensed evoked response (or an epoch-averaged evoked response) over consecutive unit times (e.g., consecutive data sampling intervals) within the time window. The signal power may be measured as an area under the curve (AUC) of the sensed evoked response (or the epoch-averaged evoked response) within the time window. In some examples, the signal analyzer circuit 1426 may generate a spatial distribution of extracted signal features across sensing locations of the sensing electrodes.

The signal analyzer circuit 1426 may compare the filtered ERs, or signal features or a spatial distribution of signal features derived therefrom, to one or more states or conditions of the patient. In some examples, the states or conditions are used to adjust the neuromodulation parameters. In some examples, the signal analyzer circuit 1426 may accumulate the sensed ERs, and therefore determined states or conditions, obtained in multiple stimulation-ER recording sessions during which stimulation pulses are delivered via a particular stimulating electrode with varying stimulation parameter settings (e.g., stimulation amplitude, frequency, or pulse width), and compare the accumulated ERs (or signal features or a spatial distribution of signal features derived from the filtered ERs) to previously stored states or conditions. In an example, the states or conditions of the patient may be user-provided or loaded into the system for easy recognition. In another example, the state or condition may be associated with a target ER or target ER feature template representing a patient-specific ER feature or a population-based ER feature to electrostimulation of the neural target. In an example, the target ER template may be used to adjust the neuromodulation therapy to relieve symptoms or other goals such as co-therapy (e.g., leads that inject drugs or light), and side-effect avoidance. The signal analyzer circuit 1426 may determine a distribution of sensed ER features, compare the determined distribution of sensed ERs to the corresponding states or conditions to determine whether to adjust the neuromodulation therapy.

The therapy controller 1428 may generate a control signal to the electrostimulator 1440 to adjust the thresholds of the neuromodulation therapy based on the determined state or condition. The electrostimulator 1440 may be configured to deliver electrical stimulation according to a stimulation setting. The electrical stimulation may be delivered using a monopolar (far-field) or a bipolar (near-field) configuration. Examples of the therapy setting may include, electrode selection and configuration, stimulation parameter values including, for example, amplitudes, pulse width, frequency, pulse waveform, active or passive recharge mode, ON time, OFF time, therapy duration, and fractionalization, among others. In an example, the therapy controller 1428 may be implemented as a proportional integral (PI) controller, a proportional-integral-derivative (PID) controller, or other suitable controller that takes the comparison of the sensed ERs (or features or a distribution of the features thereof) to the corresponding states or conditions as a feedback on the adjustment of stimulation settings.

The electrostimulator 1440 may be an implantable module, such as incorporated within the IPG 102 in FIG. 1. Alternatively, the electrostimulator 1440 may be an external stimulation device, such as incorporated with the ETS 40. In some examples, the user may choose to either send a notification (e.g., to the RC 45 or a smartphone with the patient) for a therapy reminder, or to automatically initiate or adjust neuromodulation therapy in accordance with the adjusted therapy setting. If an automatic therapy initiation is selected, the electrostimulator 1440 may deliver stimulation in accordance with the adjusted therapy setting.

In some examples, the therapy controller 1428 may generate a recommendation to the user to adjust the device setting (e.g., a programmable parameter of the electrostimulator 1440) to cause the sensed ERs to align with or to compare more favorably to one or more states or conditions of the patient. In some embodiments, the display may provide a suggestion to the user to adjust stimulation parameters to cause the since developed responses to more favorably compare to the state or condition or a transition to a different state or condition. In some examples, the therapy controller 1428 may determine or modify therapeutic stimulation settings based on the sense ERs or features thereof and the corresponding determined state or condition of the patient. The electrostimulator 1440 may deliver therapeutic stimulation (e.g., DBS) in accordance with the determined or modified therapeutic stimulation settings.

In some examples, the user interface 1450 allows a physician to remotely review therapy settings and treatment history, consult with the patient to obtain information including pain relief and SCS-related side effects or symptoms, perform remote programming of the electrostimulator 1440, or provide other treatment options to the patient. The user interface 1450 may allow a user (e.g., the patient, the physician managing the patient, or a device expert) to view, program, or modify a device setting. For example, the user may use one or more user interface (UI) control elements to provide or adjust values of one or more device parameters, or select from a plurality of pre-defined stimulation programs for future use. Each stimulation program may include a set of stimulation parameters with respective pre-determined values. In some examples, the user interface 1450 may include a display to display textually or graphically information provided by the user via an input unit, and device settings including, for example, feature selection, sensing configurations, signal pre-processing settings, therapy settings, optionally with any intermediate calculations. In an example, the user interface 1450 may present to the user an “optimal” or improved therapy setting, such as determined based on a closed-loop or adaptive feedback control of electrostimulation based on a selected evoked response signal feature, in accordance with various embodiments discussed in this document. In some examples, the user may use the user interface 1450 to provide feedback on a neuromodulation therapy, including, for example, side effects or symptoms arise or persist associated with the neurostimulation, or severity of the symptom or a side effect.

As used herein, the term “machine readable medium” may include any medium that is capable of storing, encoding, or carrying instructions for execution by a processing device or machine and that causes the processing device or machine to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

Various examples are illustrated in the figures above. One or more features from one or more of these examples may be combined to form other examples.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A method, comprising:
use a model to determine neural tissue of a patient activated by medication administered to a patient;
determine a stimulation parameter from a stimulation parameter set that activates the neural tissue during delivery of electrostimulation;
map the neural tissue activated by the medication to the stimulation parameter; and
select the stimulation parameter from the stimulation parameter set in response to the medication being associated with the patient.

2. The method of claim 1, comprising using the mapping to determine whether to increase or decrease a medication dosage and change a stimulation parameter of the stimulation parameter set.

3. The method of claim 1, comprising determining:
a map of stimulation clinical effect data (CED) based on the stimulation parameter set without administration of medication; and
a map of medication CED based on medicating the patient without applying stimulation.

4. The method of claim 3, wherein the map of the medication CED is simulated based on the neural tissue activated by the medication.

5. The method of claim 4, wherein the map of the medication CED and the neural tissue activated is compared to equivalent anatomical locations of electrical stimulation.

6. The method of claim 3, wherein the map of medication CED is an estimated map of medication CED that is predicated on a medication state without the patient being in the medication state.

7. The method of claim 3, wherein the map of stimulation CED is an estimated map of stimulation CED that is estimated for a medication state without the patient being in the medication state.

8. The method of claim 3, comprising adjusting the map of the medication CED in response to a period of time occurring associated with a decrease in medication effectiveness post-administration or nonlinear or non-monotonous dynamics.

9. The method of claim 3, comprising adjusting the map of the stimulation CED in response to a period of time occurring associated with a decrease or increase in medication effectiveness.

10. The method of claim 3, wherein the determining of the map of the medication CED is performed without having the patient washing out of medication.

11. The method of claim 1, comprising generating the model of neural tissue activated by medication based on at least one of pharmacodynamics, medication kinetics, an affect on neural pathways of the patient, patient medical history, and a symptom or side effect profile of the patient.

12. The method of claim 1, comprising acquiring medication data associated with the patient.

13. The method of claim 12, wherein the medication data comprises a medication type, a medication dosage amount, and pharmacodynamics associated with a medication for administering to the patient.

14. The method of claim 12, wherein the model is generated based on the medication data to simulate a medication state of the patient.

15. The method of claim 12, wherein the medication data is acquired based on medications prescribed to the patient.

16. The method of claim 12, wherein the medication data is acquired in response to dosing the patient and acquiring the medication data at a peak of medication dosage.

17. A non-transitory computer-readable medium storing instructions executable by a processor to:
use a number of models to each determine a set of neural tissue of a patient activated by a corresponding medication;
determine a stimulation parameter from a stimulation parameter set that activates each of the corresponding neural tissue;
generate a number of maps that each map the neural tissue activated by the corresponding medications to their respective stimulation parameters;
select respective stimulation parameters from the stimulation parameter set in response to the corresponding medications being associated with the patient; and
adjust the respective stimulation parameters based on a determined schedule for the medications.

18. A system, comprising:
a processing system configured for use to:
use a model to determine neural tissue activated by medication administered to a patient;
determine a stimulation parameter from a stimulation parameter set that activates the neural tissue during delivery of electrostimulation;
map the neural tissue activated by the medication to a stimulation parameter set providing similar change in neural activity of the patient; and
select the stimulation parameter from the stimulation parameter set in response to the medication being associated with the patient.

19. The system of claim 18, wherein the processing system is configured for use to program at least one medical device with the selected stimulation parameter to deliver the electrostimulation.

20. The system of claim 18, wherein the processing system is configured to increase or decrease a medication dosage and increase or decrease a stimulation parameter of the stimulation parameter set based on a change in the activation of the neural tissue.
